(19) United States
(12) Patent Application Publication
(10) Pub. No.: US 2026/0092298 A1
(43) Pub. Date: Apr. 2, 2026

(54) BLEOMYCIN PRODUCING STRAIN AND APPLICATION THEREOF

(71) Applicant: Hubei Honch Pharmaceutical Co.,Ltd, Huanggang (CN)

(72) Inventors: Renwen ZHANG, Huanggang (CN); Wenkai ZHANG, Huanggang (CN); Wei ZHENG, Huanggang (CN); Jianye GAN, Huanggang (CN); Hua DONG, Huanggang (CN); Liyun KE, Huanggang (CN); Li TIAN, Huanggang (CN); Na JIANG, Huanggang (CN); Fang HONG, Huanggang (CN)

(73) Assignee: Hubei Honch Pharmaceutical Co.,Ltd, Huanggang (CN)

(21) Appl. No.: 19/273,407

(22) Filed: Jul. 18, 2025

(30) Foreign Application Priority Data
Sep. 30, 2024 (CN) 202411381946.7

(51) Int. Cl. C12P 21/00; C12N 1/20

(52) U.S. Cl. CPC C12P 21/005; C12N 1/20

(57) ABSTRACT

A production strain of bleomycin and its applications are provided. Through mutagenesis, a strain, Streptomyces mobaraensis BL-G2-202402, which exhibits high resistance to bleomycin, is obtained. Its deposit number is CCTCC NO: M 20241482. By adding bleomycin-backbone amino acids to its fermentation medium formula, the fermentation titer of bleomycin was increased, and the ratio of the main components bleomycin A2 and B2 was affected. When adding 0.5 g/L of bromogeramine and 2.5-10 g/L of oxygen-enhancing agent, the yield of bleomycin could be significantly increased. When using dried bacterial residue to replace part of the nitrogen source in the medium, it was found that fermentation titer increased if freeze-dried bacterial residue replace 10-40% of the corn steep powder as the nitrogen source for bleomycin fermentation.

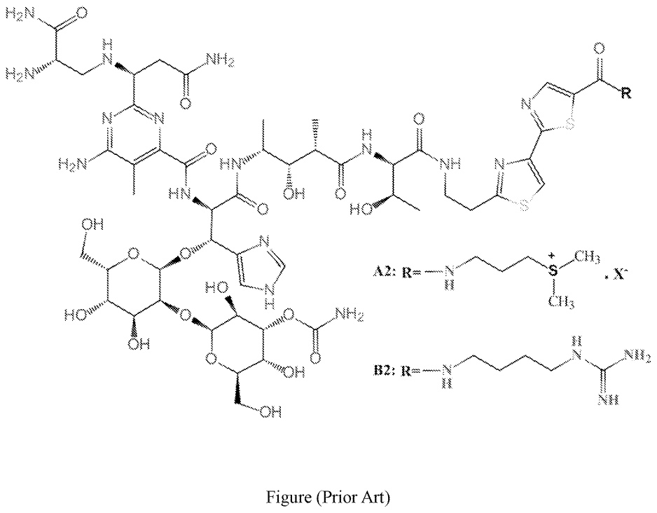

### CROSS REFERENCE TO THE RELATED APPLICATIONS

This application is based upon and claims priority to Chinese Patent Application No. 202411381946.7, filed on Sep. 30, 2024, the entire contents of which are incorporated herein by reference.

### TECHNICAL FIELD

The present invention belongs to the technical field of microbial fermentation, specifically relating to a production strain of bleomycin and its applications.

### BACKGROUND

Bleomycin (Blm) is a water-soluble glycopeptide antibiotic. It was first isolated from the fermentation broth of Streptomyces verticillus by Umezawa et al. in 1967 (see “Studies on Bleomycin”, published on Cancer, May 1967). According to the different Rf values in paper chromatography, bleomycin can be divided into group A and group B. The main difference among its homologues lies in the different C-terminal amine structures on the side chain of the aglycone ligand. Currently, the imported bleomycin injection drug BLENOXANE® used clinically mainly contains bleomycin A2 (60%) and B2 (30%) as the main components (the structure is shown in FIGURE). During chemotherapy, bleomycin does not cause significant bone marrow suppression. Therefore, it can be used in combination with other antibiotics to treat malignant germ-cell carcinomas, head and neck cancers, skin cancers, Hodgkin's lymphoma, squamous-cell carcinomas, etc. For example, the combination of bleomycin, etoposide, and cisplatin can treat 90% of testicular cancers.

Given the complexity of the bleomycin structure and the difficulty of chemical synthesis, currently, the bi-fermentation method is still the main industrial production method for bleomycin A2 and B2. Domestic research on bleomycin mainly focuses on the optimization of fermentation conditions and the identification of new analogues. There are few domestic enterprises with the qualification to produce bleomycin drugs, and there is a great demand for the active pharmaceutical ingredient in the market.

The fermentation of bleomycin is a highly oxygen-consuming process. Dissolved oxygen is crucial to the success and yield of bleomycin production. In addition, the ratio of A2 and B2 needs to be adjusted close to the optimal ratio of 2:1 through fermentation process conditions and fermentation formula adjustments, which saves a large amount of cost for subsequent purification processes

Bleomycin mainly exists in the fermentation broth. The conventional collection method is as follows: first, adjust the pH of the original bleomycin fermentation broth to 6.0 with acetic acid, and then perform solid-liquid separation by centrifugation or plate-frame filtration. Collect the liquid, and then enrich, recover, and purify bleomycin through methods such as resin adsorption to obtain bleomycin. However, the bacteria after solid-liquid separation can only be treated as solid waste. How to turn this waste into treasure and achieve “green production” is also an urgent problem that we need to solve.

### SUMMARY

This application takes a strain of Streptomyces mobaraensis BL-G2-202402 preserved by the applicant as the research object. Based on the research, bleomycin-backbone amino acids were added to its fermentation medium formula to investigate the impact of the addition of backbone amino acids on the fermentation titer of bleomycin and the ratio of the main components A2 and B2. Meanwhile, previous research has found that the production process of bleomycin has particularly high requirements for oxygen. In order to enable existing equipment to meet the production requirements of bleomycin, substances that increase cell permeability and substances that increase the oxygen-carrying capacity of the fermentation broth were added simultaneously to the bleomycin fermentation medium to examine the influence of these substances on the fermentation level of bleomycin and the ratio of the main components A2 and B2, providing a reference for the pilot-scale test in a fermenter.

On the one hand, the present invention provides a strain of Streptomyces mobaraensis by mutagenesis, specifically the strain is Streptomyces mobaraensis BL-G2-202402, deposited at the China Center for Type Culture Collection (CCTCC) on Jul. 4, 2024. The deposit number is CCTCC NO: M 20241482. And The address of CCTCC is Wuhan University, Wuhan, China, with a postal code of 430072.

On the other hand, the present invention provides an application of the strain Streptomyces mobaraensis BL-G2-202402 described above, especially its application in the production of bleomycin.

Furthermore, the application method for producing bleomycin using the strain Streptomyces mobaraensis BL-G2-202402 is as follows:

Inoculating the seed liquid into the fermentation medium at an inoculation amount of 10 20% (v:v), with a shaker rotation speed of 250-270 rpm, a cultivation temperature of 28-29° C., and a cultivation period of 8-10 d.

The fermentation medium includes: glucose 8-12 g/L, corn starch 30-50 g/L, sucrose 20-30 g/L, soybean cake powder 30-40 g/L, corn steep powder 15-20 g/L, zinc sulfate 1-1.5 g/L, copper sulfate 0.02-0.05 g/L, and pH 7.0.

Furthermore, the fermentation medium is also added with 1 g/L of L-tryptophan, 1 g/L of DL-methionine, and 1 g/L of L-cysteine.

Furthermore, the fermentation medium is also added with 0.5 g/L of bromogeramine and 2.5-10 g/L of an oxygen-enhancing agent. The oxygen-enhancing agent can be any one of liquid paraffin, n-dodecane, or n-hexadecane. Preferably, the fermentation medium is added with 0.5 g/L of bromogeramine and 5 g/L of n-hexadecane.

Furthermore, 10-40% (w/w) of the corn steep powder in the fermentation medium is replaced with the dried bacterial residue of Streptomyces mobaraensis BL-G2-202402.

Furthermore, the preparation method of the dried bacterial residue is as follows:

Centrifuging the bleomycin fermentation broth of Streptomyces mobaraensis BL-G2-202402 to collect the bacterial sludge. Washing the bacterial sludge repeatedly with water, then collecting and drying the residual bacteria. After drying, collecting the dried bacterial residue respectively and crush it for later use.

Furthermore, the drying method is a high-temperature drying or a freeze-drying.

Furthermore, conditions of the high-temperature drying conditions is 60-80° C. for 2-3 h.

Furthermore, conditions of the freeze-drying process are as follows:
1. Pre-freezing: Pre-freeze at −40° C. for 3 h, and then start vacuum-pumping.
2. Sublimation: It is divided into three stages. The first stage is the rapid heating stage. The material temperature rises from −40° C. to −30° C., and the heating rate is controlled at 10° C. per hour. The second stage is the temperature-maintaining stage. The material temperature is maintained between −30° C. and −25° C. for 10 hours. The third stage is the rapid heating stage. The material temperature rises from −25° C. to 20° C., and the heating rate is controlled at 5° C. per hour (turn on automatic heating).
3. Collect the freeze-dried bacterial residue and crush it for standby.

### Beneficial Effects

Through mutagenesis, the present invention obtained a strain, Streptomyces mobaraensis BL-G2-202402, which has high resistance to bleomycin, and then applied it to the production of bleomycin. The strain Streptomyces mobaraensis BL-G2-202402 can grow normally on a plate containing 500 mg/L of bleomycin. Adding 100-300 mg/L of bleomycin to the fermentation system also has no obvious impact on its growth and metabolism.

By adding bleomycin-backbone amino acids to the fermentation medium formula and optimizing their combination, the present invention found that when the medium is added with 1 g/L of L-tryptophan, 1 g/L of DL-methionine, and 1 g/L of L-cysteine, the total titer can reach 309.21 mg/L, and the ratio of bleomycin A2:B2 can reach 1.77:1.

The present invention added substances that increase cell permeability and substances that increase the oxygen-carrying capacity of the fermentation broth into the fermentation medium simultaneously, and investigated the influence of these substances on the fermentation level of bleomycin and the ratio of the main components bleomycin A2 and B2. It was finally determined that adding 0.5 g/L of bromogeramine and 2.5-10 g/L of an oxygen-enhancing agent can increase the titer of bleomycin. Especially for the medium added with 0.5 g/L of bromogeramine and 5 g/L of n-hexadecane, the titer of bleomycin can reach 385.96 mg/L.

The present invention replaced part of the nitrogen source in the fermentation medium with dried bacterial residue and found that freeze-dried bacterial residue can replace 10-40% of the corn steep powder as the nitrogen source for bleomycin fermentation. The fermentation titer increased slightly, with the maximum increase reaching 7.87%, and there was no significant change in the ratio of A2 to B2.

### BRIEF DESCRIPTION OF THE DRAWINGS

FIGURE shows the chemical structure of bleomycin.

In the FIGURE, X⁻ represents an acid-radical ion. For example, in the bleomycin sulfate molecule, X⁻ represents a sulfate ion, and in the bleomycin hydrochloride molecule, X⁻ represents a chloride ion.

### DETAILED DESCRIPTION OF THE EMBODIMENTS

To make the objectives, technical solutions, and advantages of this patent clearer, the following provides a more detailed description of this patent in combination with specific embodiments. It should be understood that the specific embodiments described herein are only used to explain this patent and are not intended to limit the present invention.

### Methods for Treating Bleomycin Fermentation Broth and HPLC Detection Used in the Present Disclosure:

Sample treatment: After the bleomycin fermentation is completed, the pH value in the medium is neutral or weakly alkaline, while bleomycin is relatively stable under weakly acidic conditions. To improve the stability of bleomycin in the fermentation broth, before determining the concentration of bleomycin, adjust the pH of the fermentation broth to around 6.0 with a 10% acetic acid solution. Take 10 mL of the fermentation broth and centrifuge it (12000 r/min, 10 min). Take 1 mL of the supernatant and filter it through a 0.22-μm water-based microporous membrane for liquid chromatography determination.

Chromatographic analysis conditions: The preparation method of the aqueous phase is to dissolve 7.53 g of sodium hexanesulfonate and 3.72 g of disodium ethylenediaminetetraacetate in 1 L of ultrapure water, add 4.6 mL of glacial acetic acid (chromatographic grade), and adjust the pH to 4.3 with ammonia water. The preparation method of the organic phase is methanol:acetonitrile=7:3. Use an LC Column 250×4.6 mm, 5-μm C18 chromatographic column, with a column temperature of 40° C., a flow rate of 1.0 mL/min, an ultraviolet detection wavelength of 294 nm. The liquid chromatography gradient elution program is shown in Table 1.

TABLE 1
Liquid chromatography gradient elution programembodiment

| Time (min) | aqueous phase (%) | organic phase (%) |
|---|---|---|
| 0 | 70 | 30 |
| 15 | 68 | 32 |
| 35 | 60 | 40 |
| 36 | 70 | 30 |
| 50 | 70 | 30 |

### Formula:

$$Total Titer ( mg / L ) = \frac{{A}_{sample} \times {C}_{contrast} \times P \times 0.94}{{A}_{contrast}} \times 1000$$

$$Titer of Bleomycin {A}_{2} = \frac{peak area of {A}_{2}}{peak area of {A}_{2} + peak area of {B}_{2}} \times Total Titer$$

$$Titer of Bleomycin {B}_{2} = \frac{peak area of {B}_{2}}{peak area of {A}_{2} + peak area of {B}_{2}} \times Total Titer$$

In the formula:
Aₛₐₘₚₗₑ - - - The sum of the peak areas of bleomycin A2 and bleomycin B2 in the chromatogram of the sample test solution;
A_{contrast} - - - The sum of the peak areas of bleomycin A2 and bleomycin B2 in the chromatogram of the contrast solution;
C_{contrast} - - - The concentration of the contrast solution, mg/ml;
P - - - The purity of the bleomycin sulfate contrast/reference substance, %;
0.94 - - - Conversion coefficient; the ratio of the molecular weight of bleomycin (about 1418) to the molecular weight of bleomycin sulfate (about 1515);
1000 - - - Unit conversion coefficient.

### Remarks

1. The bleomycin reference substance/contrast substance used in this application is bleomycin sulfate with a purity of 97% (the ratio of A2:B2 is 2.23:1).
2. Treatment of the bleomycin reference substance: Prepare bleomycin sulfate at a concentration of 1000 mg/L with sterile normal saline. After preparation, filter it through a 0.22-micron microporous membrane for sterilization.

The present invention will be further explained and illustrated through specific embodiments as follows.

### Example 1 Comparison of Resistance to Bleomycin Between Bleomycin-Producing Strain BL-G2-202402 and the Original Strain BL-G1-1

Strain BL-G2-202402 is obtained by mutagenesis from Streptomyces mobaraensis BL-G1-1 se|t-collected and preserved in our laboratory. It was obtained by mutagenesis using ARTP room-temperature plasma technology and combined with resistance screening, resulting in a strain with high resistance to bleomycin. The original strain and the resistant strain were successively inoculated into solid media and liquid fermentation media containing different concentrations of bleomycin. The growth status and colony size of the strains on the solid media with different concentrations of bleomycin were observed. The titers of the two strains in the fermentation media added with different concentrations of bleomycin were determined to judge the difference in the feedback ability of the resistant strain to its own metabolites, thus predicting its potential high-yield ability.

### 1. Medium

Slant Medium: Glucose 4 g/L, yeast powder 4 g/L, malt extract powder 10 g/L, calcium carbonate 2 g/L, agar 18 g/L.
Seed Medium: Glucose 2.5 g/L, malt extract powder 10 g/L, yeast extract powder 2 g/L, corn steep powder 0.5 g/L, pH 7.0.
Fermentation Medium: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, pH 7.0.

### 2. Experimental Methods

### 2.1 Comparison of Resistance Cultivation of Bleomycin-Producing Strains

The bleomycin-producing strains BL-G1-1 and BL-G2-202402 were respectively inoculated onto solid media (same as the slant medium) containing 100-700 mg/L of bleomycin, and cultured at 28° C. for 7 d. The growth status and colony size of the strains on the solid media with different concentrations of bleomycin were observed. The specific results are shown in Table 2.

TABLE 2
Influence of Adding Bleomycin to the Solid Medium of Bleomycin on Bleomycin Yield

| | | addition amount of Bleomycin | | | | | | |
|---|---|---|---|---|---|---|---|---|
| | contrast | 100 mg/L | 200 mg/L | 300 mg/L | 400 mg/L | 500 mg/L | 600 mg/L | 700 mg/L |
| Strain BL - | Normal | Normal | Normal | Normal | Beable to | small | No | No |
| G1 - 1 | growth | growth | growth | growth | grow | amount of | growth | growth |
| | | | | | | growth | | |
| | Large | Large | Large | Large | Small | Small | | |
| | colony | colony | colony | colony | colony | colon | | |
| Strain BL - | Normal | Normal | Normal | Normal | Normal | Beable to | Beable to | small |
| G2 - 202402 | growth | growth | growth | growth | growth | grow | grow | amount of |
| | | | | | | | | growth |
| | Large | Large | Large | Large | Large | Large | Small | Small |
| | colony | colony | colony | colony | colony | colony | colony | colony |

Remark: The added substance is bleomycin sulfate, and the above-mentioned addition amount is converted to the amount of bleomycin.

As can be seen from Table 2, the resistance of the screened resistant strain to bleomycin is significantly higher than that of the original strain, which remarkably increases the possibility of improving the yield of bleomycin.

### 2.2 Shake-Flask Fermentation Method

The bleomycin-producing strains BL-G1-1 and BL-G2-202402 were respectively inoculated onto 60 mL/250 mL egg-plant bottle slant medium, and cultured at 28° C. for 7 d for standby. Using a sterilized inoculation needle, 1 cm² of slant seeds were scraped off and inoculated into seed medium bottles with a liquid volume of 30 mL/250 mL. Cultivate at 28° C. and 270 rpm for about 48 hours. When the seed liquid is viscous, and a large amount of hyphae can be observed under a microscope with a long-strip network-shaped mycelial morphology, the seed liquid can be transferred to a fermentation bottle.

The liquid volume of the fermentation bottle is 30 mL/250 mL, the inoculation amount is 10%, the shaker rotation speed is 270 rpm, the cultivation temperature is 28° C., and the cultivation period is 9 d. When the growth is normal, the color of the fermentation broth is gray. Meanwhile, under microscopic examination, the hyphae are slender, network-shaped, deeply stained, and there are no contaminating bacteria.

The seed liquids of the above two strains were respectively transferred to fermentation media added with different concentrations of bleomycin. After cultivation according to the above-mentioned control parameters, the titer of the fermentation broth was determined to infer whether the resistant strain can resist the feedback inhibition of its own metabolites. The specific results are shown in Table 3.

TABLE 3
Influence of Adding Bleomycin to the Liquid
Medium of Bleomycin on Bleomycin Yield

| addition amount | 100 | 200 | 300 | 400 |
|---|---|---|---|---|
| of Bleomycin | mg/L | mg/L | mg/L | mg/L |
| mycelial morphology of | Normal | Normal | No | No |
| strain BL - G1 - 1 | growth | growth | growth | growth |
| Titer of strain BL - G1 - 1 | 168.32 | 85.64 | 0 | 0 |
| mycelial morphology of | Normal | Normal | Normal | Abnormal |
| strain BL - G2 - 202402 | growth | growth | growth | growth |
| Titer of strain | 218.75 | 216.58 | 221.64 | 109.68 |
| BL - G2 - 202402 | | | | |

Remark: The bleomycin added to the fermentation broth is bleomycin sulfate, and the above-mentioned addition amount is converted to the amount of bleomycin; the fermentation titers of strain BL-G1-1/strain BL-G2-202402 are the titers after deducting the added bleomycin sulfate.

As can be seen from Table 3, adding 100 mg/L of bleomycin to the fermentation broth of strain BL-G1-1 has no effect on its growth and metabolism, while adding 200 mg/L of bleomycin has a significant impact on the growth and metabolism of strain BL-G1-1. For strain BL-G2-202402, adding 100-300 mg/L of bleomycin to the fermentation broth has no obvious impact on its growth and metabolism, and when the addition amount reaches 400 mg/L, it has a significant impact on its growth and metabolism.

### Example 2. Influence of Adding Bleomycin-Backbone Amino Acids to the Fermentation Medium on the Titer of Bleomycin Fermentation Broth and the Ratio of A2 and B2

The bleomycin molecule contains a typical hybrid peptide-polyketide structure and a disaccharide unit. The peptide-polyketide backbone molecule is composed of 9 molecules of amino acids (serine, asparagine, histidine, threonine, cysteine, alanine, β-alanine), 1 molecule of acetic acid, and 2 molecules of S-adenosylmethionine (AdoMet).

This example is used to determine the influence of backbone amino acids L-tryptophan and DL-methionine, as well as their combinations with other amino acids, on improving the titer of bleomycin and the ratio of A2 and B2.

### 1. Medium

Slant Medium (g/L): Glucose 4 g/L, yeast powder 4 g/L, malt extract powder 10 g/L, calcium carbonate 2 g/L, agar 18 g/L.
Seed Medium: Glucose 2.5 g/L, malt extract powder 10 g/L, yeast extract powder 2 g/L, corn steep powder 0.5 g/L, pH 7.0.
   Fermentation Medium (w/v):
Control 0: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, pH 7.0.
Control 1: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L. DL-methionine 1 g/L, pH 7.0.
Formula 1: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-serine 1 g/L, pH 7.0.
Formula 2: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-asparagine 1 g/L, pH 7.0.
Formula 3: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-histidine 1 g/L, pH 7.0.
Formula 4: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-threonine 1 g/L, pH 7.0.
Formula 5: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, pH 7.0.
Formula 6: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-alanine 1 g/L, pH 7.0.
Formula 7: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, β-alanine 1 g/L, pH 7.0.

### 2. Shake-Flask Fermentation Method

Inoculate the bleomycin-producing strain BL-G2-202402 onto a 60 mL/250 mL egg-plant bottle slant medium, and culture it at 28° C. for 7 d for standby. Use a sterilized inoculation needle to scrape off 1 cm² of slant seeds and inoculate them into a seed-medium bottle with a liquid volume of 30 mL/250 mL. Cultivate at 28° C. and 270 rpm for about 48 hours. When the seed liquid is viscous and a large amount of hyphae can be observed under a microscope with a long-strip network-shaped mycelial morphology, the seed liquid can be transferred to a fermentation bottle.

Inoculate the above-mentioned seed liquid into fermentation-medium bottles containing 7 different structural amino acids respectively, with the media without added amino acids and without added structural amino acids as controls. The liquid volume of the fermentation bottle is 30 mL/250 mL, the inoculation amount is 10%, the shaker rotation speed is 270 rpm, the cultivation temperature is 28° C., and the cultivation period is 9 d. When the growth is normal, the color of the fermentation broth is gray. Meanwhile, under microscopic examination, the hyphae are slender, network-shaped, deeply stained, and there are no contaminating bacteria.

### 3. Experimental Results

After the fermentation is completed, determine the contents of bleomycin A2 and B2 in the fermentation broth. The results are shown in Table 4:

TABLE 4
Influence of Amino - acid Combinations on the Fermentation
Level of Bleomycin and the Ratio of A2 to B2

| | Content of A | Content of B | Total titer | Ratio of A2 to |
|---|---|---|---|---|
| No. | 2(mg/L) | 2(mg/L) | (mg/L) | B2 |
| Control 0 | 97.28 | 106.9 | 204.18 | 0.91:1 |
| Control 1 | 107.69 | 123.78 | 231.47 | 0.87:1 |
| Formula 1 | 85.05 | 113.41 | 198.46 | 0.75:1 |
| Formula 2 | 95.11 | 100.12 | 195.23 | 0.95:1 |
| Formula 3 | 102.29 | 111.19 | 213.48 | 0.92:1 |
| Formula 4 | 107.50 | 125.01 | 232.51 | 0.86:1 |
| Formula 5 | 197.58 | 111.63 | 309.21 | 1.77:1 |
| Formula 6 | 87.55 | 121.60 | 209.15 | 0.72:1 |
| Formula 7 | 134.24 | 28.50 | 162.74 | 4.71:1 |

From the above results, it can be seen that adding 1 g/L of L-tryptophan, 1 g/L of DL-methionine, and 1 g/L of L-cysteine simultaneously to the basic formula not only significantly improves the fermentation level of bleomycin but also remarkably improves the ratio of A2 and B2.

### Example 3 Influence of Oxygen-Enhancing Agents and Surfactants on the Fermentation Level of Bleomycin

Previous research has found that adding a low-concentration bromogeramine solution has no negative impact on the growth and metabolism of the bleomycin-producing bacteria. However, adding bromogeramine can improve the permeability of the cell membrane and enhance the level of cell metabolism. Adding an oxygen-enhancing agent can increase the dissolved oxygen level in the fermentation broth and enhance the metabolic capacity of microbial cells, thereby improving the fermentation level of bleomycin. In this study, an oxygen-enhancing agent and the surfactant bromogeramine were added to the bleomycin fermentation medium in combination to investigate their impact on the yield of bleomycin.

### 1. Medium

Slant Medium (g/L): Glucose 4 g/L, yeast powder 4 g/L, malt extract powder 10 g/L, calcium carbonate 2 g/L, agar 18 g/L.
Seed Medium (g/L): Glucose 2.5 g/L, malt extract powder 10 g/L, yeast extract powder 2 g/L, corn steep powder 0.5 g/L, pH 7.0.

### Fermentation Medium Formulas:

Control 0: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, pH 7.0.
Control 1: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, pH 7.0.
Control 2: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 1 g/L, pH 7.0.
Formula 1: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, liquid paraffin 2.5 g/L, pH 7.0.
Formula 2: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, n-dodecane 2.5 g/L, pH 7.0.
Formula 3: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, n-hexadecane 2.5 g/L, pH 7.0.
Formula 4: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, liquid paraffin 5 g/L, pH 7.0.
Formula 5: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, n-dodecane 5 g/L, pH 7.0.
Formula 6: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, n-hexadecane 5 g/L, pH 7.0.
Formula 7: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, liquid paraffin 10 g/L, pH 7.0.
Formula 8: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, n-dodecane 10 g/L, pH 7.0.
Formula 9: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, n-hexadecane 10 g/L, pH 7.0.

### 2. Shake-Flask Fermentation Method

Inoculate the bleomycin-producing strain BL-G2-202402 onto a 60 mL/250 mL egg-plant bottle slant medium, and culture it at 28° C. for 7 d for standby. Use a sterilized inoculation needle to scrape off 1 cm² of slant seeds and inoculate them into a seed-medium bottle with a liquid volume of 30 mL/250 mL. Cultivate at 28° C. and 270 rpm for about 48 hours. When the seed liquid is viscous and a large amount of hyphae can be observed under a microscope with a long-strip network-shaped mycelial morphology, the seed liquid can be transferred to a fermentation bottle.

Inoculate the above-mentioned seed liquid into fermentation-medium bottles containing 11 different combinations of different concentrations of bromogeramine and combinations of bromogeramine and oxygen-enhancing agents. The medium without added bromogeramine and oxygen-enhancing agent is used as a control. The liquid volume of the fermentation bottle is 30 mL/250 mL, the inoculation amount is 10%, the shaker rotation speed is 270 rpm, the cultivation temperature is 28° C., and the cultivation period is 9 d. When the growth is normal, the color of the fermentation broth is gray. Meanwhile, under microscopic examination, the hyphae are slender, network-shaped, deeply stained, and there are no contaminating bacteria.

### 3. Experimental Results

After the fermentation is completed, determine the contents of bleomycin A2 and B2 in the fermentation broth. The results are shown in Table 5 below:

TABLE 5
Influence of the Combination of Bromogeramine and Oxygen
- enhancing Agents on the Fermentation Level of Bleomycin

| | Content of A | Content of B | Total titer | Ratio of A2 to |
|---|---|---|---|---|
| No. | 2(mg/L) | 2(mg/L) | (mg/L) | B2 |
| Control 0 | 199.52 | 111.46 | 310.98 | 1.79:1 |
| Control 1 | 216.33 | 122.91 | 339.24 | 1.76:1 |
| Control 2 | 150.87 | 65.60 | 216.47 | 2.30:1 |
| Formula 1 | 215.71 | 123.26 | 338.97 | 1.75:1 |
| Formula 2 | 216.11 | 123.49 | 339.60 | 1.75:1 |
| Formula 3 | 220.23 | 122.35 | 342.58 | 1.80:1 |
| Formula 4 | 217.43 | 120.80 | 338.23 | 1.80:1 |
| Formula 5 | 233.86 | 133.63 | 367.49 | 1.75:1 |
| Formula 6 | 246.62 | 139.34 | 385.96 | 1.77:1 |
| Formula 7 | 231.14 | 128.41 | 359.55 | 1.80:1 |
| Formula 8 | 233.35 | 132.58 | 365.93 | 1.76:1 |
| Formula 9 | 247.18 | 135.07 | 382.25 | 1.83:1 |

As can be seen from the results in Table 5, adding 0.5 g/L of bromogeramine can improve the fermentation level of bleomycin. The combined addition of 0.5 g/L of bromogeramine and 2.5-10 g/L of liquid paraffin, n-dodecane, or n-hexadecane can all improve the fermentation level of bleomycin, while the ratio of A2 and B2 does not change significantly. Overall, the optimal combination is determined to be 0.5 g/L of bromogeramine+5 g/L of n-hexadecane.

### Example 4 Application of Using Bleomycin Fermentation Bacterial Residue to Replace Nitrogen Source 1. Medium

Slant Medium (g/L): Glucose 4 g/L, yeast powder 4 g/L, malt extract powder 10 g/L, calcium carbonate 2 g/L, agar 18 g/L.
Seed Medium (g/L): Glucose 2.5 g/L, malt extract powder 10 g/L, yeast extract powder 2 g/L, corn steep powder 0.5 g/L, pH 7.0.
Fermentation Medium: Glucose 10 g/L, corn starch 45 g/L, sucrose 25 g/L, soybean cake powder 35 g/L, corn steep powder 17.5 g/L, zinc sulfate 1.22 g/L, copper sulfate 0.05 g/L, L-tryptophan 1 g/L, DL-methionine 1 g/L, L-cysteine 1 g/L, bromogeramine 0.5 g/L, n-hexadecane 5 g/L, pH 7.0.

### 2. Experimental Methods

### 2.1 Preparation of Dried Bleomycin Bacteria

After the bleomycin fermentation in Formula 6 of Example 3 was completed, the pH of the fermentation broth was adjusted to 6.0 with glacial acetic acid, and solid-liquid separation was carried out by high-speed centrifugation. The centrifuged bacterial sludge was washed 3 times repeatedly with deionized water of 3-fold weight. The specific operation was as follows: collect the centrifuged bacterial sludge, add deionized water of 3-fold weight of the bacterial sludge, stir evenly, and then centrifuge. Repeat this process three times and collect the bacterial sludge.

The bacterial sludge was dried by high-temperature drying and low-temperature drying respectively. For high-temperature drying, it was dried at 60° C. for 2.5 hours. For low-temperature drying, freeze-drying was used. The process parameters of the drying process were as follows: (1) Pre-freezing: Pre-freeze at −40° C. for 3 h and then start vacuum-pumping. (2) Sublimation: It was divided into three stages. The first stage was the rapid heating stage. The material temperature rose from −40° C. to −30° C., and the heating rate was controlled at 10° C. per hour. The second stage was the temperature-maintaining stage. The material temperature was maintained between −30° C. and −25° C. for 10 hours. The third stage was the rapid heating stage. The material temperature rose from −25° C. to 20° C., and the heating rate was controlled at 5° C. per hour (turn on automatic heating). (3) After freeze-drying was completed, collect the dried bacterial residue.

The bacterial residues dried by different drying methods were crushed respectively for later use.

2.2 Results of the Experiment on Replacing Nitrogen Source with Bleomycin Bacterial Residue

After partially replacing the nitrogen source (soybean cake powder/corn steep powder) in the above-mentioned fermentation medium with high-temperature and low-temperature dried bacterial residues respectively, bleomycin fermentation was carried out using the same shake-flask fermentation method as in Example 3. The results are shown in Table 6 and Table 7.

TABLE 6
Experiment on Replacing Nitrogen Source with
High - temperature Dried Bacterial Residue

| the replaced | proportion of | Content of A | Content of B | Total titer | Ratio of |
|---|---|---|---|---|---|
| Nitrogen Source | being replaced | 2(mg/L) | 2(mg/L) | (mg/L) | A2 to B2 |
| contrast | 0 | 243.14 | 136.6 | 379.74 | 1.78:1 |
| soybean cake | 10% | 224.23 | 126.04 | 350.27 | 1.78:1 |
| powder | 20% | 221.62 | 113.65 | 335.27 | 1.95:1 |
| | 40% | 216.55 | 85.59 | 302.14 | 2.53:1 |
| | 60% | 131.19 | 23.10 | 154.29 | 5.68:1 |
| corn steep | 10% | 257.12 | 140.50 | 397.62 | 1.83:1 |
| powder | 20% | 258.67 | 143.70 | 402.37 | 1.80:1 |
| | 40% | 251.84 | 140.70 | 392.54 | 1.79:1 |
| | 60% | 228.93 | 136.26 | 365.19 | 1.68:1 |

Conclusion: High-temperature dried bacterial residue cannot replace soybean cake powder as the nitrogen source for bleomycin fermentation. High-temperature dried bacterial residue can replace 10-40% of corn steep powder as the nitrogen source for bleomycin fermentation. The fermentation titer increased slightly, and the ratio of A2 to B2 did not change significantly.

TABLE 7
Experiment on Replacing Nitrogen Source with
Low - temperature Dried Bacterial Residue

| the replaced | proportion of being | Content of A | Content of B | Total titer | Ratio of A2 |
|---|---|---|---|---|---|
| Nitrogen Source | replaced | 2(mg/L) | 2(mg/L) | (mg/L) | to B2 |
| contrast | 0 | 244.88 | 137.58 | 382.46 | 1.78:1 |
| soybean cake | 10% | 243.50 | 137.57 | 381.07 | 1.77:1 |
| powder | 20% | 241.20 | 130.38 | 371.58 | 1.85:1 |
| | 40% | 223.77 | 118.39 | 342.16 | 1.89:1 |
| | 60% | 108.62 | 18.92 | 127.54 | 5.74:1 |
| corn steep powder | 10% | 258.61 | 153.94 | 412.55 | 1.68:1 |
| | 20% | 263.33 | 143.90 | 407.23 | 1.83:1 |
| | 40% | 255.95 | 142.19 | 398.14 | 1.80:1 |
| | 60% | 229.99 | 128.48 | 358.47 | 1.79:1 |

Conclusion: Low-temperature dried bacterial residue can replace 10-20% of soybean cake powder as the nitrogen source for bleomycin fermentation. At the same time, low-temperature dried bacterial residue can also replace 10-40% of corn steep powder as the nitrogen source for bleomycin fermentation. The fermentation titer increased slightly, with the maximum increase reaching 7.87%, and the ratio of A2 to B2 did not change significantly.

The above-described embodiments merely represent several implementation modes of the present invention. Their descriptions are relatively specific and detailed, but they should not be construed as limitations to the scope of the patent. It should be noted that for those of ordinary skill in the art, without departing from the concept of this patent, the above-mentioned implementation modes can be modified, combined, and improved in several ways, and these all fall within the protection scope of this patent. Therefore, the protection scope of this patent should be subject to the claims.

What is claimed is:

1-10. (canceled)

11. A strain of Streptomyces mobaraensis, wherein the strain is Streptomyces mobaraensis BL-G2-202402, with a deposit number: CCTCC NO: M 20241482.

12. An application of the strain of the Streptomyces mobaraensis BL-G2-202402 according to claim 11 in a production of bleomycin.

13. The application according to claim 12, wherein a method for producing the bleomycin comprises: inoculating seed liquid of the Streptomyces mobaraensis BL-G2-202402 into a fermentation medium at an inoculation amount of 10-20%, with a shaker rotation speed of 250-270 rpm, a cultivation temperature of 28-29° C., and a cultivation period of 8-10 d.

14. The application according to claim 13, wherein the fermentation medium comprises: glucose 8-12 g/L, corn starch 30-50 g/L, sucrose 20-30 g/L, soybean cake powder 30-40 g/L, corn steep powder 15-20 g/L, zinc sulfate 1-1.5 g/L, and copper sulfate 0.02-0.05 g/L, with pH of 7.0.

15. The application according to claim 14, wherein the fermentation medium further comprises 1 g/L of L-tryptophan, 1 g/L of DL-methionine, and 1 g/L of L-cysteine.

16. The application according to claim 15, wherein the fermentation medium further comprises 0.5 g/L of bromogeramine and 2.5-10 g/L of an oxygen-enhancing agent; and the oxygen-enhancing agent is one of liquid paraffin, n-dodecane, or n-hexadecane.

17. The application according to claim 16, wherein the fermentation medium comprises 0.5 g/L of the bromogeramine and 5 g/L of the n-hexadecane.

18. The application according to claim 17, wherein in the fermentation medium, 10-40% of the corn steep powder is replaced with a dried bacterial residue of the Streptomyces mobaraensis BL-G2-202402.

19. The application according to claim 18, wherein the dried bacterial residue is prepared by the following steps:
centrifuging a bleomycin fermentation broth of the Streptomyces mobaraensis BL-G2-202402 to obtain bacterial sludge, collecting the bacterial sludge, washing the bacterial sludge repeatedly with water to obtain residual bacteria, and then collecting and drying the residual bacteria to obtain a dried bacterial residue; and after drying, collecting the dried bacterial residue respectively and crushing the dried bacterial residue for later use.

20. The application according to claim 19, wherein the drying is a high-temperature drying or a freeze-drying.
